(19) United States
(12) Patent Application Publication
(10) Pub. No.: US 2022/0346521 A1
(43) Pub. Date: Nov. 3, 2022

(54) NAIL GRINDER

(71) Applicant: HAPPY UNITOYS CO., LIMITED, Shenzhen (CN)

(72) Inventor: Qinsheng Li, SHENZHEN (CN)

(21) Appl. No.: 17/329,310

(22) Filed: May 25, 2021

(30) Foreign Application Priority Data
Apr. 29, 2021 (CN) 202110472716.1

(51) Int. Cl. A45D 29/05

(52) U.S. Cl. CPC A45D 29/05; A45D 2200/205

(57) ABSTRACT

Provided is a nail grinder that includes a housing, a grinding member, a dust suction fan, a driving member, an ultraviolet lamp, and a first illumination lamp. The housing is provided with a grinding chamber, a dust collection chamber, and a fan installation chamber, which are connected in sequence. The grinding member is rotatably arranged in the grinding chamber. The dust suction fan is rotatably arranged in the fan installation chamber. The driving member is used to drive the grinding member and the dust suction fan to rotate. The ultraviolet lamp is arranged on the housing and used for emitting ultraviolet light. The first illumination lamp is arranged on the housing, and the light-emitting side of the illumination lamp is arranged to face toward the grinding member. The technical solution of this disclosure enriches the functions of the nail grinder.

### CROSS-REFERENCE TO RELATED APPLICATIONS

This application claims the benefit and priority of China patent application No. 202110472716.1 filed on Apr. 29, 2021, disclosure of which is hereby incorporated by reference in its entirety.

### TECHNICAL FIELD

This disclosure relates to the technical field of pet nail grinding, and more particularly relates to a nail grinder.

### BACKGROUND

In order to get rid of loneliness or pursue entertainment, people start to keep pets for accompany. Cats and dogs are often popular for their cuteness and loyalty. However, untrained cats and dogs are easy to urinate everywhere at home, and urine stains cannot be easily tracked out by the human eye after air-drying. In addition, cats and dogs grow nails just like humans. Pointed nails can easily scratch the pet owner or damage the furniture, so the pet owner needs to trim the pet's nails regularly.

The related art pet nail grinder needs to grind the pet's nails in a relatively bright environment. In addition, the related art pet nail grinder cannot be used to track the location of pet urine stains, nor can it collect the scraps produced during the process of grinding the pet's nails. The scraps produced are scattered around and are likely to remain on the pet owner's clothes or be inhaled by the pet owner, so that the bacteria on the scraps are easy to parasitize in or on the pet owner's body, affecting the pet owner's physical and mental health.

### SUMMARY

It is therefore a main objective of the present disclosure to provide a nail grinder, which aims to solve the technical problem of the current nail grinders have only a single function.

In order to achieve the above objective, the nail grinder provided by the present disclosure includes a housing, a grinding member, a dust suction fan, a driving member, an ultraviolet lamp, and a first illumination lamp. The housing is provided with a grinding chamber, a dust collection chamber, and a fan installation chamber, which are connected in sequence. The grinding member is rotatably arranged in the grinding chamber. The dust suction fan is rotatably arranged in the fan installation chamber. The driving member is used to drive the grinding member and the dust suction fan to rotate. The ultraviolet lamp is arranged on the housing and used for emitting ultraviolet light. The first illumination lamp is arranged on the housing, and the light-emitting side of the illumination lamp is arranged to face toward the grinding member.

In some embodiments, the grinding chamber, the dust collection chamber and the fan installation chamber are sequentially connected along a length of the housing.

In some embodiments, the housing includes an upper cover and an outer casing, where the upper cover is provided at one end of the outer casing and defines the grinding chamber and the dust collection chamber, and the outer casing is hollow thus defining the fan installation chamber.

In some embodiments, there is provided a fan blade baffle between the dust collection chamber and the fan installation chamber, and the fan blade baffle is provided with an air inlet connecting the dust collection chamber with the fan installation chamber. The first illumination lamp is arranged on a side of the fan blade baffle adjacent to the dust collection chamber, and the upper cover is provided with a first light admission opening at a position corresponding to the light-emitting side of the first illumination lamp.

In some embodiments, an end of the upper cover facing away from the outer casing is provided with a grinding port, and the grinding member partially protrudes from the grinding port.

In some embodiments, the grinding port includes a first opening and a second opening, and the size of the first opening is larger than the size of the second opening.

In some embodiments, the driving member includes a motor that is provided in the fan installation chamber and that is arranged on a side of the dust suction fan facing away from the grinding member. The output shaft of the motor extends from the fan installation chamber to the grinding chamber, and is drivingly connected with both the dust suction fan and the grinding member.

In some embodiments, the dust suction fan includes a centrifugal fan blade and an axial fan blade that are sequentially connected in the length of the housing. The air inlet side of the centrifugal fan blade faces the dust collection chamber, and the air outlet side of the centrifugal fan blade faces the air inlet side of the axial fan blade. The air outlet side of the axial fan blade faces the motor, and the peripheral wall of the outer casing is provided with an air outlet facing the air outlet side of the axial fan blade.

In some embodiments, the ultraviolet lamp is arranged at an end of the housing that faces away from the grinding member, and the light-emitting side of the ultraviolet lamp faces away from the grinding member.

In some embodiments, there is further provided an LED projection lamp at the end of the housing facing away from the grinding member, and the LED projection lamp includes a LED lamp bead and a film that is provided with a preset pattern, and the light-emitting side of the LED lamp bead faces towards the film to illuminate a light spot with the preset pattern; and/or

there is further provided a laser lamp on the end of the housing facing away from the grinding member; and/or

there is further provided a second illumination lamp on the end of the housing facing away from the grinding member.

In some embodiments, the driving member further includes a PCB board arranged in the fan installation chamber, and the PCB board is electrically connected to each of the motor, the ultraviolet lamp, the first illumination lamp, the LED projection lamp, the laser lamp, and the second illumination lamp, so as to control the turning on and off of the motor, the ultraviolet lamp, the first illumination lamp, the LED projection lamp, the laser lamp, and the second illumination lamp.

In some embodiments, the housing is provided with a function button corresponding to the position of the PCB board, and the function button includes at least one of a motor speed adjustment button or a light adjustment button.

In some embodiments, the driving member further includes a power supply provided in the fan installation chamber, and the power supply is used to supply power to the motor, the PCB board, the ultraviolet lamp, the first illumination lamp, the LED projection lamp, the laser lamp, and the second illumination lamp.

According to the nail grinder provided by the technical solutions of the present disclosure, a grinding chamber, a dust collection chamber, and a fan installation chamber are defined in the housing. The dust suction fan is driven by the driving member, so that when the grinding member in the grinding chamber rotates thus grinding the pet's nails, the dust suction fan would rotate to drive the air in the grinding chamber to flow from the grinding chamber to the dust collection chamber. Then the duct collection chamber is used to filter and store the nail scraps in the airflow, so as to realize the collection and storage of nail scraps by the nail grinder. Further, by providing the first illumination lamp on the housing to emit light toward the grinding member, the user can observe the pet nails, effectively avoiding excessive grinding that may damage the blood-lined parts of the pet nails, thus improving the reliability of the nail grinder. Furthermore, an ultraviolet lamp is provided on the housing, so that the user can find the location of pet urine stains in the home based on the ultraviolet light emitted by the ultraviolet lamp, and accordingly perform operations such as currency verification or disinfection. In view of the above, compared with the related art nail grinder, the nail grinder of the present application has the advantages of providing richer functions and more hygienic use.

### BRIEF DESCRIPTION OF DRAWINGS

For a clearer understanding of the technical solutions that are used in the embodiments according to the present disclosure or that are used in the related art, hereinafter the drawings that are required for the description of the embodiments disclosed herein or the related art will be briefly introduced. Apparently, the drawings in the following description merely represent some embodiments of the present disclosure, and for those having ordinary skill in the art, other drawings may also be obtained based on the structures or arrangement shown in these drawings without investing creative efforts.

FIG. 1 is a schematic diagram illustrating a nail grinder according to an embodiment of the present disclosure.

FIG. 2 is a schematic diagram illustrating the structure of the nail grinder of FIG. 1 from another perspective.

FIG. 3 is a schematic diagram illustrating the structure of the nail grinder of FIG. 1 from yet another perspective.

FIG. 4 is a schematic cross-sectional view of the nail grinder of FIG. 1 with part of the structure hidden out of view.

FIG. 5 is a schematic diagram illustrating the structure of the nail grinder of FIG. 1 with the housing hidden.

FIG. 6 is a schematic cross-sectional view of the housing of the nail grinder of FIG. 1.

### DESCRIPTION OF REFERENCE SIGNS

1. Housing; 11. Upper cover; 111. Grinding member; 112, Dust collection chamber; 113. Grinding port; 113a. First opening; 113b. Second opening; 114. First light-admission opening; 12. Outer casing; 121. Fan installation chamber; 122. Second light-admission opening; 123. Air outlet; 13. Fan blade baffle; 131. Air inlet; 2. Polishing member; 3. Dust suction fan; 31. Centrifugal fan blade; 32. Axial fan blade; 4. Driving member; 41. Motor; 42, PCB board; 43. Power supply; 5. Ultraviolet lamp; 6. First light illumination lamp; 7. Second light illumination lamp; 8. Lampshade; 9. Function button; 91. Motor speed adjustment button; 92. Light adjustment button; 10. LED projection lamp; 101. LED lamp bead; 102. Film; 100. Laser lamp

The realization of the objectives, functional features, and advantages of the present disclosure will be described in further detail in connection with the embodiments and with reference to the drawings.

### DETAILED DESCRIPTION OF ILLUSTRATIVE EMBODIMENTS

Hereinafter, technical solutions in the embodiments according to the present disclosure will be described in a definite and comprehensive manner in connection with the accompanying drawings in the embodiments according to the present disclosure. Apparently, the embodiments described herein are only a part, rather than all of the embodiments in accordance with the present disclosure. Based on the embodiments of the present disclosure, all other embodiments obtained by those having ordinary skill in the art without investing creative efforts shall all fall in the scope of protection of the present disclosure.

It should be noted that any directional or orientational indication (such as up, down, left, right, front, back . . . ) as used in the embodiments disclosed herein is merely used to explain the relative positional relationships and movement of the components in a specific posture (as shown in the figures). Should the particular posture changes, the directional or orientational indication would also change accordingly.

In addition, terms “first”, “second”, or the like as used herein are merely used for illustrative purposes, and shall not be construed as indicating relative importance or implicitly indicating the number of technical features specified. Thus, unless otherwise specified, the features defined by “first” and “second” may explicitly or implicitly include at least one of such feature. In addition, the technical solutions according to the various embodiments can be combined with each other, but it must be based on what can be achieved by a person having ordinary skill in the art. When the combination of technical solutions is conflicting or cannot be achieved, it should be considered that such a combination of technical solutions does not exist, nor does it fall in the scope of protection of the present disclosure.

The present disclosure provides a nail grinder, which is mainly used to grind the nails of a pet. Of course, the nail grinder can also be used to grinder the nails of a human.

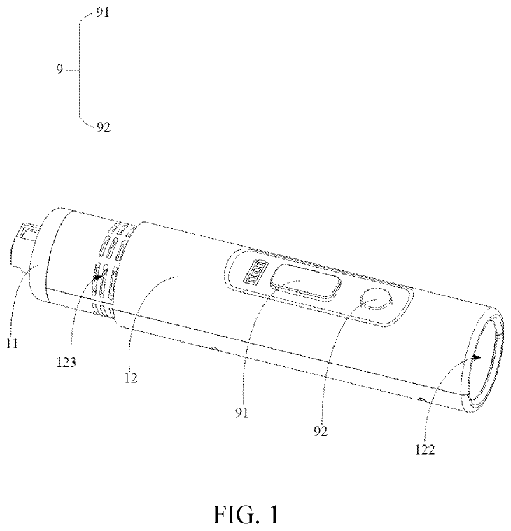

In the embodiments according to the present disclosure, as illustrated in FIGS. 1, 2, 5, and 6, the nail grinder includes a housing 1, a grinding member 2, a dust suction fan 3, a driving member 4, an ultraviolet lamp 5, and a first illumination lamp 6. The housing 1 is provided with a grinding chamber 111, a dust collection chamber 112, and a fan installation chamber 121, which are connected in sequence. The grinding member 2 is rotatably arranged in the grinding chamber 111. The dust suction fan 3 is rotatably arranged in the fan installation chamber 121. The driving member 4 is used to drive the grinding member 2 and the dust suction fan 3 to rotate. The ultraviolet lamp 5 is arranged on the housing 1 and used for emitting ultraviolet light. The first illumination lamp 6 is arranged on the housing 2, and the light-emitting side of the illumination lamp 6 is arranged to face toward the grinding member 2.

The first illumination lamp 6 may be an LED lamp that can emit visible light.

More particularly, the dust collection chamber 112 may have an air inlet and an air outlet, where the air inlet of the dust collection chamber 112 communicates with the grinding chamber 111, and the air outlet of the dust collection chamber 112 communicates with the fan installation chamber 121. The dust suction fan 3 is installed in the fan installation chamber 121, the air inlet end of the dust suction fan 3 is communicated with the air outlet of the dust collection chamber 112, and the air outlet end of the dust suction fan 3 is communicated with the outside of the nail grinder. In other words, when the driving member 4 drives the dust suction fan 3 into rotation, the air flows from the grinding chamber 111 to the dust collection chamber 112, and then to the fan installation chamber 121, and then is discharged to the outside of the nail grinder. Correspondingly, a filter screen may be provided between the dust collection chamber 112 and the fan installation chamber 121, where the filter screen is used to filter nail scraps in the air, so that the nail scraps can be collected and stored in the dust collection chamber 112.

It is appreciated that the related art nail grinder has the technical problem of not being able to collect nail scraps. Regarding this problem, according to the nail grinder provided by the technical solutions of the present disclosure, a grinding chamber 111, a dust collection chamber 112, and a fan installation chamber 121 are defined in the housing 1. The dust suction fan 3 is driven by the driving member 4, so that when the grinding member 2 in the grinding chamber 111 rotates thus grinding the pet's nails, the dust suction fan 3 would rotate to drive the air in the grinding chamber 111 to flow from the grinding chamber 111 to the dust collection chamber 112. Then the duct collection chamber 112 is used to filter and store the nail scraps in the airflow, so as to realize the collection and storage of nail scraps by the nail grinder 1. Further, by providing the first illumination lamp 6 on the housing 1 to emit light toward the grinding member 2, the user can observe the pet nails, effectively avoiding excessive grinding that may damage the blood-lined parts of the pet nails, thus improving the reliability of the nail grinder. Furthermore, an ultraviolet lamp 5 is provided on the housing 1, so that the user can find the location of pet urine stains in the home based on the ultraviolet light emitted by the ultraviolet lamp 5, and accordingly perform operations such as currency verification or disinfection. In view of the above, compared with the related art nail grinder, the nail grinder of the present application has the advantages of providing richer functions and more hygienic use.

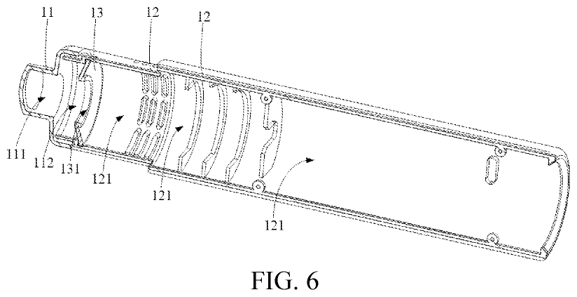

In some embodiments, as illustrated in FIG. 6, the housing 1 includes an upper cover 11 and an outer casing 12. The upper cover 11 is arranged at one end of the outer casing 12, and defines the grinding chamber 111 and the dust collection chamber 112. The outer casing 12 is hollowed to form the fan installation chamber 121. Specifically, in the present application, the outer casing 12 may be designed in a cylindrical shape, so that the arc-shaped outer surface of the outer casing 12 fits the palm of the user more easily, which is beneficial to enhance the user's hand-held experience. Furthermore, a filter screen may be provided between the upper cover 11 and the outer casing 12, so that when nail scraps flow from the inside of the upper cover 11 to the inside of the outer casing12 along with the airflow, they can be filtered out by the filter screen and stored in the dust collection chamber 112 inside the upper cover 11.

In some embodiments, there is provided a fan blade baffle 13 between the dust collection chamber 112 and the fan installation chamber 121, and the fan blade baffle 13 is provided with an air inlet 131 connecting the dust collection chamber 112 with the fan installation chamber 121. The first illumination lamp 6 is arranged on a side of the fan blade baffle 13 adjacent to the dust collection chamber 112, and the upper cover 11 is provided with a first light admission opening 114 at a position corresponding to the light-emitting side of the first illumination lamp 6. It will be appreciated that the air inlet 131 is communicated with the air outlet of the dust collection chamber 112, and the dust collection chamber 112 and the fan installation chamber 121 are separated by the fan blade baffle 13, which effectively ensures the safe operation of the dust suction fan 3 in the fan installation chamber 121. Moreover, arranging the first illumination lamp 6 on the fan blade baffle 13 is also beneficial to concealing the connection circuit of the first illumination lamp 6 and improving the reliability of the operation of the product.

In some embodiments, as illustrated in FIG. 6, the grinding chamber 111, the dust collection chamber 112 and the fan installation chamber 121 are sequentially connected along a length of the housing 1. It can be understood that this configuration can make the process of the air flowing from the grinding chamber 111 to the dust collection chamber 112 and then to the fan installation chamber 121 more smoothly, which facilitates the collection and storage of nail scraps by the nail grinder of the present application. It is worth noting that the design of the present application will not be limited to this. In other embodiments, the grinding chamber 111, the dust collection chamber 112, and the fan installation chamber 121 may also be arranged in other manners, such as but not limited to, the fan installation chamber 121 is provided on the side of the dust collection chamber 112 adjacent to the grinding chamber 111.

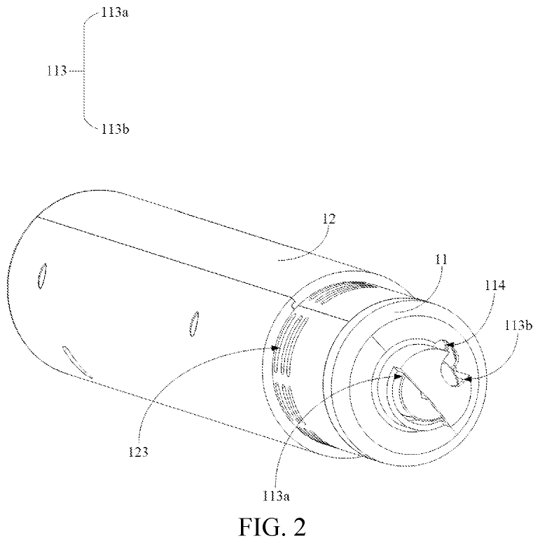

In some embodiments, as illustrated in FIG. 2, an end of the upper cover 11 facing away from the outer casing 12 is provided with a grinding port 113, and the grinding member 2 partially protrudes from the grinding port 113. It will be appreciated that a part of the grinding member 2 extends out of the grinding port 113 to rotate and grind the pet's nails, and the other part of the grinding member 2 is hidden in the grinding chamber 111 of the upper cover 11, which helps to avoid excessive exposure of the grinding member 2 to the outside which may otherwise cause accidental injury to the pet or pet owner, thereby improving the safety of the nail grinder of the present application. In addition, concealing the other part of the grinding member 2 in the grinding chamber111 of the upper cover 11 also makes it convenient for the upper cover 11 to collect the nail scraps thrown out from the grinding member 2, which is beneficial to improve the dust suction reliability of the nail grinder of the present application.

In some embodiments, as illustrated in FIG. 2, the grinding port 113 may include a first opening 113a and a second opening 113b, and the size of the first opening 113a is larger than the size of the second opening 113b. It is understandable that different pets may have different nail sizes, and the user can choose an opening of a suitable size depending on the size of the pet's nails to grind the pet's nails. In this way, the flexibility of the nail grinder of this application is improved. It should be noted that the design of the present application will not be limited to this. In other embodiments, the size of the first opening 113a may also be equal to the size of the second opening 113b, which is not limited herein.

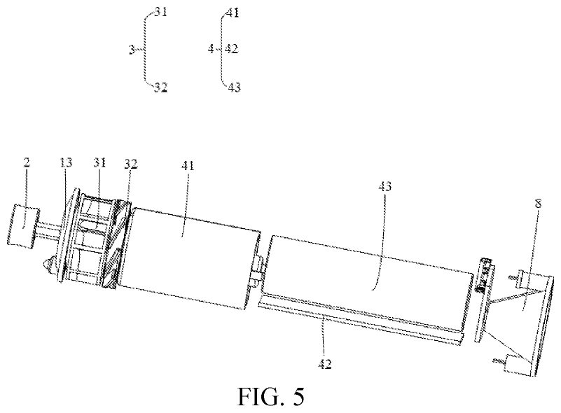

In some embodiments, as illustrated in FIG. 5, the driving member 4 includes a motor 41 that is provided in the fan installation chamber 121 and that is arranged on a side of the dust suction fan 3 facing away from the grinding member 2. The output shaft of the motor 41 extends from the fan installation chamber 121 to the grinding chamber 111, and is drivingly connected with both the dust suction fan 3 and the grinding member 2. It can be understood that, compared to the way that two motors 41 are used to drive the dust suction fan 3 and the grinding member 2 respectively, this application uses the same motor 41 to drive the dust suction fan 3 and the grinding member 2 at the same time, which can maximize the use efficiency of the motor 41, which is also conducive to the miniaturized design of the nail grinder of the present application.

In some embodiments, as illustrated in FIG. 5, the dust suction fan 3 includes a centrifugal fan blade 31 and an axial fan blade 32 that are sequentially connected in the length of the housing 1. The air inlet side of the centrifugal fan blade 31 faces the dust collection chamber 112, and the air outlet side of the centrifugal fan blade 31 faces the air inlet side of the axial fan blade 32. The air outlet side of the axial fan blade 32 faces the motor 41, and the peripheral wall of the outer casing 12 is provided with an air outlet 123 facing the air outlet side of the axial fan blade 32. It will be appreciated that the double-blade structure can effectively increase the suction power of the dust suction fan 3 and help ensure the dust collection reliability of product. In addition, the air inlet side of the axial fan blade 32 is adjacent to the air outlet side of the centrifugal fan blade 31, which can effectively increase the air outlet speed on the air outlet side of the centrifugal fan blade 31. Further, the air outlet side of the axial fan blade 32 faces the motor 41, and the air outlet 123 faces the air outlet side of the axial fan blade 32, so that when the blade 32 is discharging the air from the fan installation chamber 121, it also effectively reduces the temperature of the motor 41 at the same time.

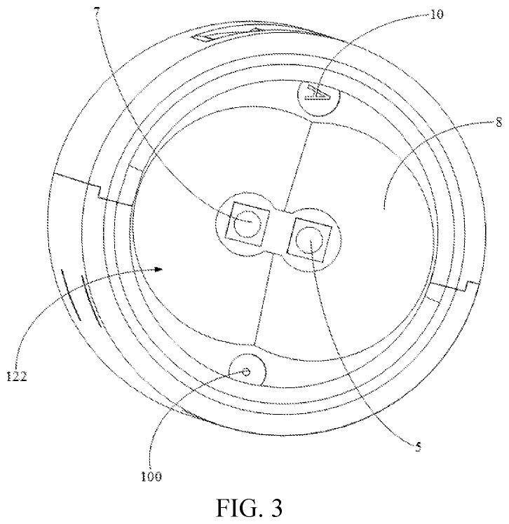

In some embodiments, as illustrated in FIG. 3, the ultraviolet lamp 5 is arranged at an end of the housing 1 that faces away from the grinding member 2, and the light-emitting side of the ultraviolet lamp 5 faces away from the grinding member 2. The end of the housing 1 facing away from the grinding member 2 is provided with a second light-admission opening 122. It can be understood that the arrangement is such that the user can hold the side wall of the outer casing 12 and conveniently irradiate the light of the ultraviolet lamp 5 to the designated area, thus facilitating the various operations such as finding the location of pet urine stains at home, verifying the genuity of currency notes, or disinfection.

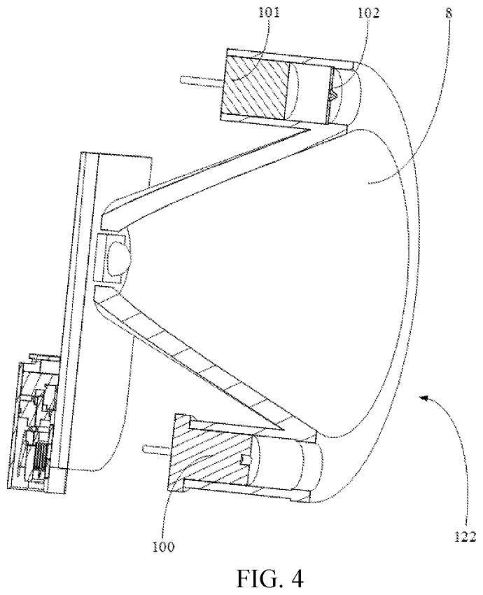

In some embodiments, as illustrated in FIGS. 3 and 4, there is further provided an LED projection lamp 10 at the end of the housing 1 facing away from the grinding member 2, and the LED projection lamp 10 includes a LED lamp bead 101 and a film 102 that is provided with a preset pattern, and the light-emitting side of the LED lamp bead 101 faces towards the film 102 to illuminate a light spot with the preset pattern. It will be appreciated that the corresponding preset pattern can be printed on the film 102. When the LED lamp bead 101 lights up and faces the patterned film 102, then the light spot with the preset pattern can be displayed on the ground, desktop and other places. As such, while enriching the functions of the nail grinder, it also facilitates the interaction between the pet owner and the pet, helping to consume the pet's physical strength. Then, when the pet's physical strength is almost consumed up, the pet's nails are started to be grinded. This helps to reduce the negative emotions of the pet during the process of grinding nails.

In some embodiments, the nail grinder may further include a micro-rotating mechanism, and the output end of the micro-rotating mechanism is drivingly connected to the film 102 to drive the film 102 to rotate, which helps to enrich the presentation effect of the preset pattern of the LED projection lamp 10.

In some embodiments, as illustrated in FIGS. 3 and 4, there may further be provided a laser lamp 100 at one end of the housing 1 facing away from the grinding member 2. It will be appreciated that the laser lamp 100 is also suitable for the pet owner to interact with the pet, which is helpful for consuming the pet's physical strength, and helps to reduce the pet's negative emotions during the nail grinding process.

In some embodiments, as illustrated in FIG. 3, there may further be provided a second illumination lamp 7 provided at the end of the housing 1 facing away from the grinding member 2. It will be appreciated that the second illumination lamp 7 of the present application is an LED lamp that can emit visible light. The second illumination lamp 7 can be used for lighting, and can also be used in conjunction with the ultraviolet lamp 5, that is, the irradiation directions of the ultraviolet rays and the light rays of the second illumination lamp 7 are the same, so that the user can know the irradiating direction of the ultraviolet rays through the visible rays of the second illumination lamp 7.

In particular, the laser lamp 100, the ultraviolet lamp 5, the LED projection lamp 10, and the second illumination lamp 7 are all arranged at the end of the housing 1 facing away from the grinding member 2. The light-emitting sides of the laser lamp 100, the ultraviolet lamp 5, the LED projection lamp 10 and the second illumination lamp 7 are all facing away from the polishing part 2. The housing 1 is provided with the second light-transmitting port 122 that allows the passage of the light of all four of the laser lamp 100, the ultraviolet lamp 5, the LED projection lamp 10, and the second illumination lamp 7. It can be understood that the four of the laser lamp 100, the ultraviolet lamp 5, the LED projection lamp 10 and the second illumination lamp 7 are arranged at the same end in the length direction of the housing 1. on the one hand, it is convenient for the user to hold the side wall of the housing 1, and orient the light-emitting sides of the laser lamp 100, the ultraviolet lamp 5, the LED projection lamp 10, and the second illumination lamp 7 to a specific place in a convenient manner, which helps to improve the user experience. On the other hand, the four of the laser lamp 100, the ultraviolet lamp 5, the LED projection lamp 10 and the second illumination lamp 7 share the same second light transmission port 122, which also facilitates the processing of the housing 1.

Furthermore, as illustrated in FIG. 3 and FIG. 4, the nail grinder may further include a lampshade 8 arranged on the ultraviolet lamp 5 and the second illumination lamp 7.

Further, as illustrated in FIGS. 3 and 4, the laser lamp 100 and the LED projection lamp 10 are respectively located on opposite sides of the lampshade 8, and the lampshade 8 is provided with a light through hole at the position corresponding to the laser lamp 100 and the LED projection lamp 10 that is used for allowing the light to pass through.

In some embodiments, as illustrated in FIG. 5, the driving member 4 may further include a PCB board 42 arranged in the fan installation chamber 121. The PCB board 42 is electrically connected to each of the motor 41, the ultraviolet lamp 5, the first illumination lamp 6, the LED projection lamp 10, and the laser lamp 100, and the second illumination lamp 7 to control the turning on and off of the motor 41, the ultraviolet lamp 5, the first illumination lamp 6, the LED projection lamp 10, the laser lamp 100, or the second illumination lamp 7. It will be appreciated that such a arrangement is beneficial to improve the intelligent control level of the nail grinder of the present application. In particular, the PCB board 42 is arranged between the second illumination lamp 7 and the motor 41 and extends along the length of the outer casing 12, so that the space utilization rate of the fan installation chamber 121 is improved.

In some embodiments, as illustrated in FIG. 1, the outer casing 12 is provided with a function button 9 corresponding to the position of the PCB board 42, and the function button 9 includes at least one of a motor 41 speed adjustment button or a light adjustment button 92. It can be understood that the function button 9 includes a motor 41 speed adjustment button and a light adjustment button 92. The motor 41 speed adjustment button can be used to adjust the speed of the motor 41. For example, by pressing the motor 41 speed adjustment button once, the motor 41 rotates at a first gear speed. Pressing the motor 41 speed adjustment button twice, the motor 41 will rotate at a second gear speed, and pressing the motor 41 speed adjustment button three times, and the motor 41 will rotate at a third gear speed, where the first gear speed, the second gear speed or the third gear speed respectively represent different rotational speeds of the motor 41. The light adjustment button 92 is used to simultaneously control the ultraviolet lamp 5, the first illumination lamp 6, the LED projection lamp 10, the laser lamp 100, and the second illumination lamp 7. The specific operation process may be such that different lamps may be turned on depending of the number of times the light adjustment button 92 is pressed.

In some embodiments, as illustrated in FIG. 5, the driving member 4 may further include a power supply 43 arranged in the fan installation chamber 121, and the power supply 43 is used to supply power to the motor 41, the PCB board 42, the ultraviolet lamp 5, the first illumination lamp 6, the second illumination lamp 7, the LED projection lamp 10, or the laser lamp 100. It will be appreciated that such an arrangement is beneficial to reduce the dependence of the nail grinder of the present application on external power supplies. In particular, the power supply 43 is arranged between the second illumination lamp 7 and the motor 41 and extends along the length of the outer casing 12, so that the space utilization rate of the fan installation chamber 121 is improved.

The foregoing merely depicts some illustrative embodiments of the present disclosure, which however are not intended to limit the scope of the present disclosure. Without departing from the inventive concept of the present disclosure, any equivalent structural changes made on the basis of the contents of the description and drawings of the present disclosure, or any direct/indirect use of the present disclosure on other related technical fields shall all be included in the scope of protection of the present disclosure.

What is claimed is:

1. A nail grinder, comprising:
a housing, defining a grinding chamber, a dust collection chamber, and a fan installation chamber that are connected in sequence;
a grinding member, rotatably arranged in the grinding chamber;
a dust suction fan, rotatably arranged in the fan installation chamber;
a driving member, configured to drive the grinding member and the dust suction fan to rotate;
an ultraviolet lamp, arranged in the housing and configured for emitting ultraviolet light; and
a first illumination lamp, arranged on the housing, wherein the first illumination lamp comprises a light-emitting side that is arranged to face toward the grinding member.

2. The nail grinder of claim 1, wherein the grinding chamber, the dust collection chamber, and the fan installation chamber are sequentially connected along a length of the housing.

3. The nail grinder of claim 2, wherein the housing includes an upper cover and an outer casing, wherein the upper cover is arranged at an end of the outer casing and defines the grinding chamber and the dust collection chamber, and the outer casing is hollow to define the fan installation chamber.

4. The nail grinder of claim 3, wherein there is provided a fan blade baffle between the dust collection chamber and the fan installation chamber, and wherein the fan blade baffle comprises an air inlet connecting the dust collection chamber with the fan installation chamber, wherein the first illumination lamp is arranged on a side of the fan blade baffle adjacent to the dust collection chamber, and the upper cover comprises a first light admission opening at a position corresponding to the light-emitting side of the first illumination lamp.

5. The nail grinder of claim 3, wherein there is defined a grinding port at an end of the upper cover facing away from the outer casing, and the grinding member partially protrudes from the grinding port.

6. The nail grinder of claim 5, wherein the grinding port comprises a first opening and a second opening, wherein the first opening has a larger size than that of the second opening.

7. The nail grinder of claim 6, wherein the driving member comprises a motor that is arranged in the fan installation chamber on a side of the dust suction fan facing away from the grinding member, wherein the motor comprises an output shaft that extends in a direction from the fan installation chamber to the grinding chamber and that is drivingly connected with each of the dust suction fan and the grinding member.

8. The nail grinder of claim 7, wherein the dust suction fan comprises a centrifugal fan blade and an axial fan blade that are sequentially connected in the length of the housing, wherein an air inlet side of the centrifugal fan blade faces the dust collection chamber, and an air outlet side of the centrifugal fan blade faces an air inlet side of the axial fan blade, and an air outlet side of the axial fan blade faces the motor, and wherein a peripheral wall of the outer casing comprises an air outlet facing the air outlet side of the axial fan blade.

9. The nail grinder of claim 1, wherein the ultraviolet lamp is arranged at an end of the housing facing away from the grinding member, and the light-emitting side of the ultraviolet lamp faces away from the grinding member.

10. The nail grinder of claim 9, wherein there is further arranged an LED projection lamp at the end of the housing facing away from the grinding member, and wherein the LED projection lamp comprises a LED lamp bead and a film that comprises a preset pattern, wherein a light-emitting side of the LED lamp bead faces towards the film to illuminate a light spot with the preset pattern.

11. The nail grinder of claim 9, wherein there is further provided a laser lamp on the end of the housing facing away from the grinding member.

12. The nail grinder of claim 9, wherein there is further provided a second illumination lamp on the end of the housing facing away from the grinding member.

13. The nail grinder of claim 11, wherein there is further provided a second illumination lamp on the end of the housing facing away from the grinding member.

14. The nail grinder of claim 10, wherein there is further provided a laser lamp on the end of the housing facing away from the grinding member.

15. The nail grinder of claim 10, wherein there is further provided a second illumination lamp on the end of the housing facing away from the grinding member.

16. The nail grinder of claim 14, wherein there is further provided a second illumination lamp on the end of the housing facing away from the grinding member.

17. The nail grinder of claim 2, wherein the ultraviolet lamp is arranged at an end of the housing facing away from the grinding member, and the light-emitting side of the ultraviolet lamp faces away from the grinding member.

18. The nail grinder of claim 3, wherein the ultraviolet lamp is arranged at an end of the housing facing away from the grinding member, and the light-emitting side of the ultraviolet lamp faces away from the grinding member.

19. The nail grinder of claim 4, wherein the ultraviolet lamp is arranged at an end of the housing facing away from the grinding member, and the light-emitting side of the ultraviolet lamp faces away from the grinding member.

20. The nail grinder of claim 5, wherein the ultraviolet lamp is arranged at an end of the housing facing away from the grinding member, and the light-emitting side of the ultraviolet lamp faces away from the grinding member.
